(19) United States
(12) Patent Application Publication
(10) Pub. No.: US 2019/0282475 A1
(43) Pub. Date: Sep. 19, 2019

(54) High-Performance Hair Treatment Agents With A Proteolipid And A Magnesium Salt

(71) Applicant: Henkel AG & Co. KGaA, Duesseldorf (DE)

(72) Inventors: Erik Schulze zur Wiesche, Hamburg (DE); Rene Krohn, Norderstedt (DE)

(73) Assignee: Henkel AG & Co. KGaA, Duesseldorf (DE)

(21) Appl. No.: 16/434,458

(22) Filed: Jun. 7, 2019

Related U.S. Application Data
(63) Continuation of application No. 15/243,579, filed on Aug. 22, 2016, now abandoned.

(30) Foreign Application Priority Data
Sep. 1, 2015 (DE) 102015216687.2

(51) Int. Cl. A61K 8/365; A61K 8/64; A61Q 5/00; A61K 8/65; A61K 8/19; A61Q 5/12

(52) U.S. Cl. CPC A61K 8/365; A61K 8/64; A61Q 5/00; A61Q 5/02; A61K 8/65; A61K 8/19; A61Q 5/12; A61Q 5/004

(57) ABSTRACT

Hair treatment agents with a pH between 3.5 and 5.5 include
a) 0.00001 to 20% by weight at least one proteolipid of the formula (I)

R′—X—R″ (I),
wherein
   R′ stands for a straight-chain or branched, saturated or unsaturated hydrocarbon group having 11 to 24 carbon atoms,
   R″ denotes a protein, a peptide, or a protein hydrolysate,
   X stands for —C(O)O— or —N⁺(R^{III}₂)—R^{IV}— or —N(R^{III})R^{IV}— or —C(O)—N(R^{V})R^{VI}—,
   R^{III} denotes —(CH₂)ₓ—CH₃ with x=0-22,
   R^{IV} denotes —CH₂—CH(OH)—CH₂— or —(CH₂)ₓ— with x=0-22, and
   R^{V} and R^{VI} independently of one another stand for —H or —(CH₂)ₓ—CH₃ with x=0-22,
with the proviso that R″ stands for keratin or a keratin hydrolysate, when X stands for —C(O)O—, and
b) 0.01 to 5.0% by weight, based on the total weight of the hair treatment agent, of magnesium citrate in a water-containing carrier.

### FIELD OF THE INVENTION

The present invention generally relates to hair treatment agents, in particular shampoos and so-called conditioners, with an active substance combination for the gentle and effective care of hair.

### BACKGROUND OF THE INVENTION

The importance of care products with the longest-lasting possible effect is increasing not least due to the great stressing of hair, for example, by coloring or permanents, as well as by cleaning the hair with shampoos and by harmful environmental factors. Care products of this type influence the natural structure and properties of hair. Thus, after such care treatments, for example, the wet and dry combability of hair, the hold, and fullness of the hair can be optimized or the hair can be protected from an increase in split ends.

It has long been customary, therefore, to subject the hair to a special aftertreatment. In this regard, the hair is treated with special active substances, for example, quaternary ammonium salts or special polymers, usually in the form of a rinse. Depending on the formulation, the combability, hold, and fullness of the hair can be improved by this treatment and the splitting rate reduced.

Multifunctional cosmetic products are also known in the prior art. These include in particular the so-called “2 in 1” shampoos, which not only clean the hair but also condition it. Such products are highly valued by the consumer, because due to their product performance they make at least one process step unnecessary, for example, the conditioning with a classic hair rinse.

Proteolipids are known as care substances in cosmetic agents, and corresponding products are widespread on the market. Cosmetic agents with proteolipids are known from WO 2011/042323 A2, which improve numerous properties of body surfaces treated with them, in particular hair, and apart from improved shine result in particular in an “impregnation” of the hair.

Similarly, products for modifying the natural hair color play a prominent role in hair cosmetics. Permanent, semipermanent, or temporary coloring systems are differentiated which are based on chemical and/or natural dyes. The hair colors produced artificially by permanent, semipermanent, or temporary coloring systems have the disadvantage, however, that they can change in an undesirable way, for example, during or after the cleaning of hair.

An “undesirable change” is understood to be the fading or bleeding and the loss of color brilliance of the hair shade achieved by the particular dyeing. Environmental effects and/or the effects of sunlight can intensify these changes still further.

The use of divalent metal salts in hair dyes to improve the durability and thereby the fastness of the color is known from EP 2438900 A1.

There continues to be a need to provide active substances or active substance combinations for hair treatment agents with good care properties, which, moreover, strengthen the adhesion of dyes to hair fibers and thus maintain the fastness of the artificially produced hair color, and in this respect to develop hair treatment agents further.

Furthermore, other desirable features and characteristics of the present invention will become apparent from the subsequent detailed description of the invention and the appended claims, taken in conjunction with this background of the invention.

### BRIEF SUMMARY OF THE INVENTION

A hair treatment agent with a pH between 3.5 and 5.5, obtainable by mixing 0.00001 to 20% by weight, based on the total weight of the hair treatment agent, of at least one proteolipid of the formula (I)

R′—X—R″ (I),

where R′ stands for a straight-chain or branched, saturated or unsaturated hydrocarbon group having 11 to 24 carbon atoms; R″ denotes a protein, a peptide, or a protein hydrolysate; X stands for —C(O)O— or —N⁺(R^{III}₂)—R^{IV}— or —N(R^{III})R^{IV}— or —C(O)—N(R^{V})R^{VI}—; R^{III} denotes —(CH₂)ₓ—CH₃ with x=0-22; R^{IV} denotes —CH₂—CH(OH)—CH₂— or —(CH₂)ₓ— with x=0-22; and R^{V} and R^{VI} independently of one another stand for —H or —(CH₂)ₓ—CH₃ with x=0-22; with the proviso that R″ stands for keratin or a keratin hydrolysate, when X stands for —C(O)O—; and 0.01 to 5.0% by weight, based on the total weight of the hair treatment agent, of magnesium citrate in a water-containing carrier.

A method for reducing and/or preventing the bleeding and/or fading of artificially produced hair colors and/or for improving the color intensity and/or the color fidelity, and for improving at least one of the properties tensile strength of keratinic fibers, in particular human hair; stabilization of the moisture balance of keratinic fibers, in particular human hair; combability of keratinic fibers, in particular human hair; increase in the contact angle between water drops and the surface of keratinic fibers, in particular human hair; reduction of the loss of elasticity of keratinic fibers, in particular human hair, in the case of damage by atmospheric effects, characterized in that a hair treatment agent with a pH between 3.5 and 5.5, obtainable by mixing 0.00001 to 20% by weight, based on the total weight of the hair treatment agent, of at least one proteolipid of the formula (I)

R′—X—R″ (I),

where R′ stands for a straight-chain or branched, saturated or unsaturated hydrocarbon group having 11 to 24 carbon atoms; R″ denotes a protein, a peptide, or a protein hydrolysate; X stands for —C(O)O— or —N⁺(R^{III}₂)—R^{IV}— or —N(R″)R^{IV}— or —C(O)—N(R^{V})R^{VI}—; R^{III} denotes —(CH₂)ₓ—CH₃ with x=0-22; R^{IV} denotes —CH₂—CH(OH)—CH₂— or —(CH₂)ₓ— with x=0-22; and R^{V} and R^{VI} independently of one another stand for —H or —(CH₂)ₓ—CH₃ with x=0-22; with the proviso that R″ stands for keratin or a keratin hydrolysate, when X stands for —C(O)O—; and 0.01 to 5.0% by weight, based on the total weight of the hair treatment agent, of magnesium citrate in a water-containing carrier, is applied to the keratinic fibers, left there for 5 seconds to 10 minutes, and then rinsed out, or is applied to the keratinic fibers and remains there.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that a combination of certain proteolipids with magnesium citrate has an especially positive effect on dyed hair treated therewith and the hair follicle.

A first subject of the present invention comprises hair treatment agents with a pH between 3.5 and 5.5, obtainable by mixing
a) 0.00001 to 20% by weight, based on the total weight of the hair treatment agent, of at least one proteolipid of the formula (I)

R′—X—R″ (I),
where
   R′ stands for a straight-chain or branched, saturated or unsaturated hydrocarbon group having 11 to 24 carbon atoms,
   R″ denotes a protein, a peptide, or a protein hydrolysate,
   X stands for —C(O)O— or —N⁺(R^{III}₂)—R^{IV}— or —N(R^{III})R^{IV}— or —C(O)—N(R^{V})R^{VI}—,
   R^{III} denotes —(CH₂)ₓ—CH₃ with x=0-22, and
   R^{IV} denotes —CH₂—CH(OH)—CH₂— or —(CH₂)ₓ— with x=0-22;
   R^{V} and R^{VI} independently of one another stand for —H or —(CH₂)ₓ—CH₃ with x=0-22;
with the proviso that R″ stands for keratin or a keratin hydrolysate, when X stands for —C(O)O—, and
b) 0.01 to 5.0% by weight, based on the total weight of the hair treatment agent, of magnesium citrate in a water-containing carrier.

Hair treatment agents in the context of the present invention are, for example, hair shampoos, hair conditioners, conditioning shampoos, hair sprays, hair rinses, hair treatments, hair packs, hair tonics, permanent wave fixing solutions, hair coloring shampoos, hair dyes, hair setting lotions, hair setting products, hair styling preparations, blow-dry wave lotions, styling mousses, hair gels, hair waxes, or combinations thereof. In view of the fact that men in particular are often reluctant to use multiple different agents and/or multiple application steps, such agents are preferred that a man uses in any event. Preferred agents therefore are shampoos, conditioning agents, or hair tonics.

The hair treatment agents include, based on their weight, 0.0001 to 20% by weight of at least one selected proteolipid.

The hair treatment agents include as a first essential ingredient at least one proteolipid of the formula (I)

R′—X—R″ (I),

where
R′ stands for a straight-chain or branched, saturated or unsaturated hydrocarbon group having 11 to 24 carbon atoms,
R″ denotes a protein, a peptide, or a protein hydrolysate,
X stands for —C(O)O— or —N⁺(R^{III}₂)—R^{IV}— or —N(R″)R^{IV}— or —C(O)—N(R^{V})R^{VI}—,
R^{III} denotes —(CH₂)ₓ—CH₃ with x=0-22, and
R^{IV} denotes —CH₂—CH(OH)—CH₂— or —(CH₂)ₓ— with x=0-22;
R^{V} and R^{VI} independently of one another stand for —H or —(CH₂)ₓ—CH₃ with x=0-22;
with the proviso that R″ stands for keratin or a keratin hydrolysate, when X stands for —C(O)O—.

Preferably, the proteolipids are used within specific amounts in the hair treatment agents. Preferred hair treatment agents include, based on their weight, 0.01 to 10% by weight, preferably 0.02 to 5% by weight, particularly preferably 0.05 to 2.5% by weight, more preferably 0.1 to 1% by weight, and in particular 0.15 to 0.5% by weight of proteolipid(s).

The R″ group in the formula (I) stands for a peptide or a protein or a protein hydrolysate. If X=—C(O)O—, R″ is selected from the group comprising keratin or keratin hydrolysate.

Preferred R″ groups are oligopeptides that have at least one amino acid sequence Glu-Glu-Glu

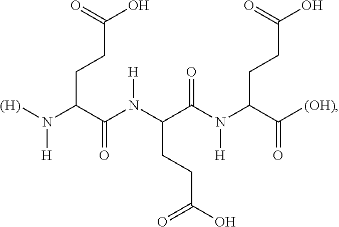

where the amino group may be present in a free or protonated form and the carboxy groups may be present in a free or deprotonated form.

In this formula, as in all formulas below, the hydrogen atom in parentheses of the amino group like the hydroxy group in parentheses of the acid function means that the groups in question may be present as such (in which case it is an oligopeptide with the particular number of amino acids as shown-in the present formula there are three amino acids) or that the amino acid sequence is present in an oligopeptide that comprises still further amino acids (depending on where the further amino acid(s) is/are bound, the components in parentheses in the above formula are replaced by the further amino acid group(s)).

Oligopeptides in the context of the present invention are amino acid condensation products which are linked acid amide-like by peptide bonds and comprise at least 3 and a maximum of 25 amino acids.

In preferred hair treatment agents of the above-described embodiment, the oligopeptide (=the R″ group) comprises 5 to 15 amino acids, preferably 6 to 13 amino acids, particularly preferably 7 to 12 amino acids, and in particular 8, 9, or 10 amino acids.

The molar mass of the proteolipid present in the agent can vary depending on whether further amino acids are bound to the Glu-Glu-Glu sequence and depending on the type of these amino acids and as a function of the selection of the R′ group and optionally R^{III} and R^{IV}. Preferred hair treatment agents are characterized in that the proteolipid has a molar mass of 1000 to 30,000 Da, preferably of 1250 to 25,000 Da, particularly preferably of 1500 to 20,000 Da, and in particular of 2000 to 15,000 Da.

Oligopeptides, which consist not just of the three glutamic acids but have further amino acids bound to this sequence, are preferably employed as the R″ group. These further amino acids are preferably selected from specific amino acids, whereas specific other representatives are less preferred.

It is preferred if the R″ group of the proteolipids used in the agents includes tyrosine.

It is preferred further if the R″ group of the proteolipids used in the agents includes leucine.

It is preferred further if the R″ group of the proteolipids used in the agents includes isoleucine.

It is preferred further if the R″ group of the proteolipids used in the agents includes arginine.

It is preferred further if the R″ group of the proteolipids used in the agents includes valine.

Amino acid sequences included as the R″ group in particularly preferred oligopeptides or in the preferred oligopeptides are described hereafter:

A particularly preferred oligopeptide additionally includes tyrosine, which is bound preferably via its acid function to the Glu-Glu-Glu sequence. Preferred hair treatment agents are therefore characterized in that the oligopeptide included as the R″ group in proteolipids of the formula (I) has at least one amino acid sequence Tyr-Glu-Glu-Glu

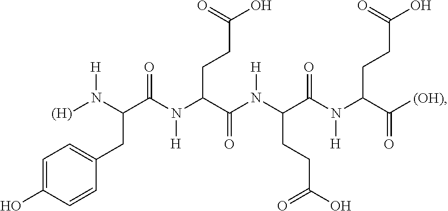

where the amino group may be present in a free or protonated form and the carboxy groups may be present in a free or deprotonated form.

A further particularly preferred oligopeptide additionally includes isoleucine, which is bound preferably via its amino function to the Glu-Glu-Glu sequence. Preferred hair treatment agents are therefore characterized in that the oligopeptide included as the R″ group in proteolipids of the formula (I) has at least one amino acid sequence Glu-Glu-Glu-Ile

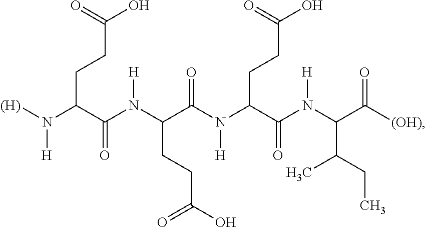

where the amino group may be present in a free or protonated form and the carboxy groups may be present in a free or deprotonated form.

Oligopeptides that have both of the aforesaid amino acids (tyrosine and isoleucine) are preferred. Particularly preferred in this case are hair treatment agents in which the oligopeptide included as the R″ group in proteolipids of the formula (I) has at least one amino acid sequence Tyr-Glu-Glu-Glu-Ile

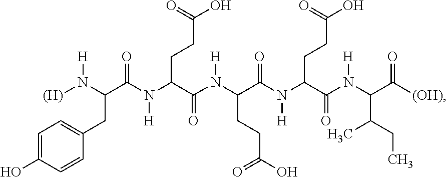

where the amino group may be present in a free or protonated form and the carboxy groups may be present in a free or deprotonated form.

Further preferred oligopeptides additionally include arginine, which is preferably present bound to isoleucine. Preferred hair treatment agents are therefore characterized in that the oligopeptide included as the R″ group in proteolipids of the formula (I) has at least one amino acid sequence Tyr-Glu-Glu-Glu-Ile-Arg

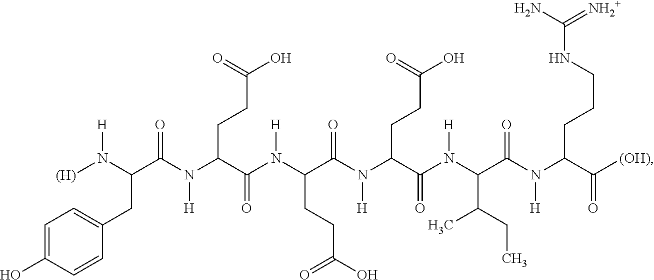

where the amino groups may be present in a free or protonated form and the carboxy groups may be present in a free or deprotonated form.

Still further preferred oligopeptides additionally include valine, which is preferably present bound to arginine. Further preferred hair treatment agents are therefore characterized in that the oligopeptide included as the R″ group in proteolipids of the formula (I) has at least one amino acid sequence Tyr-Glu-Glu-Glu-Ile-Arg-Val

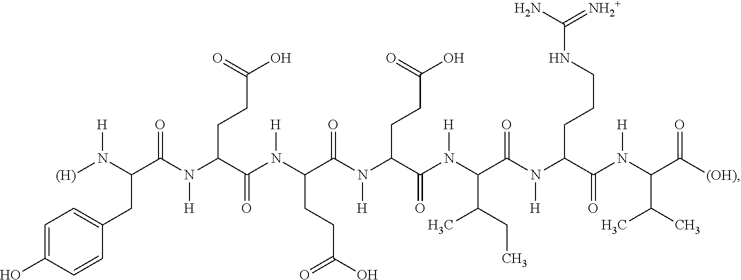

where the amino groups may be present in a free or protonated form and the carboxy groups may be present in a free or deprotonated form.

Still further preferred oligopeptides additionally include leucine, which is preferably present bound to valine. Further preferred hair treatment agents are characterized in that the oligopeptide included as the R″ group in proteolipids of the formula (I) has at least one amino acid sequence Tyr-Glu-Glu-Glu-Ile-Arg-Val-Leu

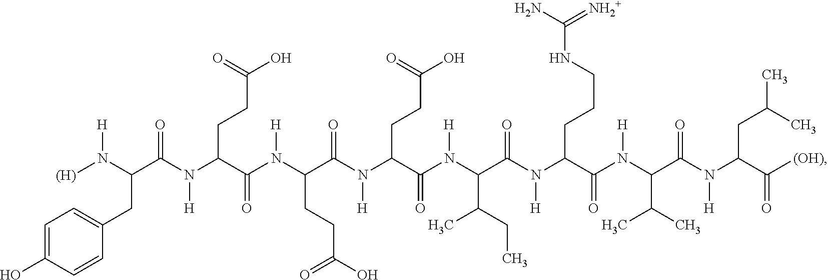

where the amino groups may be present in a free or protonated form and the carboxy groups may be present in a free or deprotonated form.

Particularly preferred oligopeptides additionally include leucine, which is preferably present bound to tyrosine. Further preferred hair treatment agents are characterized in that the oligopeptide included as the R″ group in proteolipids of the formula (I) has at least one amino acid sequence Leu-Tyr-Glu-Glu-Glu-Ile-Arg-Val-Leu

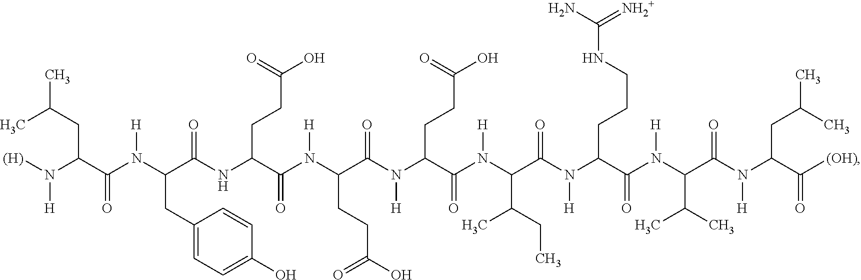

where the amino groups may be present in a free or protonated form and the carboxy groups may be present in a free or deprotonated form.

In summary, particular hair treatment agents are preferred that include at least one proteolipid of the formula (I), in which R″ has at least one amino acid sequence Leu-Tyr-Glu-Glu-Glu-Ile-Arg-Val-Leu

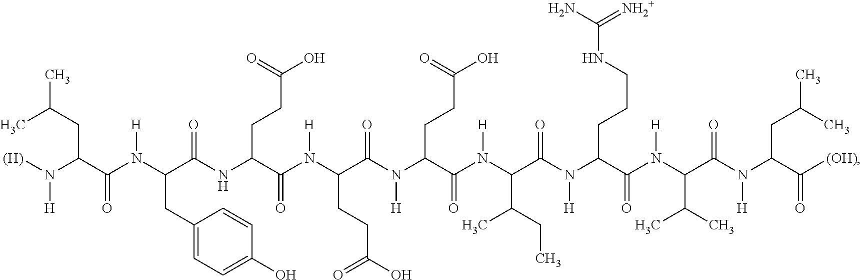

where the amino groups may be present in free or protonated form and the carboxy groups may be present in free or deprotonated form.

As already mentioned, R″ is selected from the group comprising keratin or keratin hydrolysate, if X=—C(O)O—.

In all other cases, the R″ group in formula (I) may stand for a peptide or a protein or a protein hydrolysate, protein hydrolysates being preferred. Protein hydrolysates are product mixtures obtained by acid-, base-, or enzyme-catalyzed degradation of proteins. Protein hydrolysates of both plant and animal origin may be used.

Animal protein hydrolysates are, for example, elastin, collagen, keratin, silk, and milk protein hydrolysates, which can also be present in the form of salts. Such products are sold, for example, under the trademarks Dehylan® (BASF SE), Promois® (Interorgana), Collapuron® (BASF SE), Nutrilan® (BASF SE), Gelita-Sol® (Deutsche Gelatine Fabriken Stoess & Co), Lexein® (Inolex), and Kerasol® (Croda).

It is preferred to use protein hydrolysates of plant origin, for example, soy, almond, rice, pea, potato, and wheat protein hydrolysates. Such products can be obtained, for example, under the trademarks Gluadin® (BASF SE), DiaMin® (Diamalt), Lexein® (Inolex), and Crotein® (Croda).

Preferably, independent of the choice of the X in the formula (I), the R″ group is selected from keratin or keratin hydrolysates. Preferred hair treatment agents are characterized in that they include at least one proteolipid of the formula (I) in which R″ stands for keratin or a keratin hydrolysate.

Particularly preferred R—X— groups are presented in the tables in WO 2011/042323 A2. This patent is incorporated expressly and in its entirety herein by reference. The groups disclosed therein in still further preferred embodiments of the present invention are bound to keratin or keratin hydrolysate as an R″ group.

Regardless of the choice of the —X— group in the formula (I), hair treatment agents are preferred that include at least one proteolipid of the formula (I), where R^{III} denotes —CH₃ and R^{IV} stands for —(CH₂)ₓ— with x=0, 1, 2, 3, 4, 5, 6, 7, or 8.

Further, particularly preferred hair treatment agents are characterized in that they include at least one proteolipid of the formula (I), in which X stands for —N⁺(CH₃)₂—CH₂—CH(OH)—CH₂— and R′ for —(CH₂)₁₇—CH₃.

Likewise particularly preferred hair treatment agents are characterized in that they include at least one proteolipid of the formula (I), in which X stands for —N⁺(CH₃)₂—CH₂—CH(OH)—CH₂— and R′ for —(CH₂)₁₁₋₂₃—CH₃.

Likewise further preferred hair treatment agents are characterized in that they include at least one proteolipid of the formula (I), in which X stands for —C(O)—O— and R′ for —(CH₂)₁₇—CH₃.

The agents include the proteolipid(s) preferably in an amount of 0.01 to 10.0% by weight and particularly preferably of 0.05 to 2.0% by weight, based in each case on the weight of the ready-to-use agent.

The hair treatment agents include as a second essential ingredient, based on their weight, 0.01 to 5.0% by weight of magnesium citrate.

Magnesium citrate can be present in different forms. Said forms can comprise trimagnesium dicitrate hydrate, anhydrous trimagnesium dicitrate, magnesium hydrogen citrate hydrate, or anhydrous magnesium hydrogen citrate. Particularly preferably, the employed magnesium citrate comprises anhydrous trimagnesium dicitrate (Mg₃(C₆H₅O₇)₂, CAS No.: 3344-18-1), trimagnesium dicitrate nonahydrate (Mg₃(C₆H₅O₇)₂x9H₂O, CAS No.: 153531-96-5), or magnesium hydrogen citrate (C₆H₆O₇Mg, CAS No.: 7779-25-1).

The hair treatment agents can be prepared with use of any of said forms alone or of a combination of two or more of said magnesium citrate forms. It is preferred accordingly that a hair treatment agent is obtained with use of anhydrous trimagnesium dicitrate and/or trimagnesium dicitrate nonahydrate and/or anhydrous magnesium hydrogen citrate and/or magnesium hydrogen citrate hydrate. It is particularly preferable that the hair treatment agent is obtained with use of anhydrous trimagnesium dicitrate.

The hair treatment agents include magnesium citrate preferably in an amount of 0.5 to 4.0% by weight and particularly preferably of 1.0 to 2.0% by weight, based in each case on the weight of the ready-to-use hair treatment agent. It is particularly preferred that 1.0 to 2.0% by weight, based on the weight of the ready-to-use hair treatment agent, of anhydrous trimagnesium dicitrate is used in the preparation of the hair treatment agent.

The hair treatment agent has a pH in the range of 3.5 to 5.5. Preferably, the pH is 3.75 to 5.25, more preferably 4.0 to 5.0, and in particular 4.2 to 4.7. The color modification of artificially produced hair colors after multiple treatment processes is especially low with a simultaneously good care performance, if the hair treatment agent has a pH in the range of 4.3 to 4.7.

The pH is adjusted, if necessary, by means of suitable pH adjusting agents, such as acids or bases, after the mixing of the other ingredients of the hair treatment agent.

It turned out that the use of the combination of selected proteolipids and magnesium citrate, preferably magnesium hydrogen citrate, confers excellent properties on the hair treatment agents. Thus, the color modification of artificially produced hair colors after multiple treatment processes is extremely low. In addition, the hair treatment agents impart more resilience to the hair treated therewith, which is apparent in higher tensile strengths of the keratin fibers and in a reduction of the loss of elasticity, for example, in the case of damage by atmospheric effects. Moreover, the moisture balance of the keratinic fibers is stabilized, so that the combability is improved and the aging process is delayed.

The hair treatment agents can be applied in principle to hair that was dyed with permanent, semipermanent, or temporary hair dyes. Temporary hair dyes, however, are intended to be washed out over time and/or to fade, for which reason the hair treatment agent is especially suitable for use on hair that was dyed with permanent or oxidative hair dyes.

The hair treatment agents can include further active and auxiliary substances. These are described below.

The hair treatment agents preferably include in addition at least one surfactant, wherein surface-active substances depending on the field of application are referred to as surfactants or as emulsifiers and are selected from anionic, cationic, amphoteric/zwitterionic, and nonionic surfactants and emulsifiers.

Preferred hair treatment agents are characterized in that, based on their weight, they include 0.5 to 70% by weight, preferably 1 to 60% by weight, and in particular 5 to 25% by weight of anionic and/or nonionic and/or cationic and/or amphoteric/zwitterionic surfactant(s).

All anionic surface-active substances, suitable for use on the human body, are suitable as anionic surfactants for the hair treatment agents. These are characterized by an anionic group imparting water solubility, such as, for example, a carboxylate, sulfate, sulfonate, or phosphate group, and a lipophilic alkyl group having, for instance, 8 to 30 C atoms. In addition, glycol ether or polyglycol ether groups, ester, ether, and amide groups, and hydroxyl groups can be included in the molecule.

Surface-active compounds that have at least one quaternary ammonium group and at least one —COO⁽⁻⁾ or —SO₃⁽⁻⁾ group in the molecule are called amphoteric/zwitterionic surfactants. Especially suitable zwitterionic surfactants and emulsifiers are the so-called betaines, such as the N-alkyl-N,N-dimethylammonium glycinates, for example, coco alkyl dimethylammonium glycinate and N-acyl-aminopropyl-N,N-dimethylammonium glycinates, for example, cocoacylaminopropyl dimethylammonium glycinate, and 2-alkyl-3-carboxymethyl-3-hydroxyethyl imidazolines each having 8 to 18 C atoms in the alkyl or acyl group, as well as cocoacylaminoethyl hydroxyethyl carboxymethyl glycinate. A preferred zwitterionic surfactant is the fatty acid amide derivative known under the INCI name of Cocamidopropyl Betaine.

Amphoteric/zwitterionic surfactants are also understood to be surface-active compounds that include at least one free amino group and at least one —COOH or —SO₃H group in the molecule, in addition to a C₈-C₂₄ alkyl or acyl group, and are capable of forming internal salts. Examples of suitable amphoteric/zwitterionic surfactants are N-alkyl glycines, N-alkylaminopropionic acids, N-alkylaminobutyric acids, N-alkyliminodipropionic acids, N-hydroxyethyl-N-alkylamidopropyl glycines, N-alkyltaurines, N-alkylsarcosines, 2-alkylaminopropionic acids, and alkylaminoacetic acids each having, for instance, 8 to 24 C atoms in the alkyl group.

Especially preferred amphoteric/zwitterionic surfactants are N-cocoalkylaminopropionate, cocoacylaminoethylaminopropionate, and C₁₂-C₁₈ acylsarcosine.

Nonionic surfactants and emulsifiers include as a hydrophilic group, for example, a polyol group, a polyalkylene glycol ether group, or a combination of a polyol and polyglycol ether group.

Very especially preferred are agents that include in addition fatty alcohol(s) and/or fatty alcohol alkoxylate(s), preferably C₁₂₋₂₂ fatty alcohol(s) and/or C₁₂₋₂₂ fatty alcohol ethoxylate(s) with 10 to 30 EO units, particularly preferably C₁₆₋₁₈ fatty alcohol(s) and/or C₁₆₋₁₈ fatty alcohol ethoxylate(s) with 12 to 20 EO units, preferably in amounts of 5 to 20% by weight, preferably of 7.5 to 17.5% by weight, and in particular of 10 to 15% by weight, based in each case on the weight of the hair treatment agent.

In summary, hair treatment agents are preferred that, based on their weight, include 0.1 to 20% by weight, preferably 0.25 to 17.5% by weight, and in particular 5 to 15% by weight of anionic surfactant(s), particularly preferably fatty alcohol ether sulfates of the formula

H₃C—(CH₂)ₙ—(OCH₂CH₂)ₖ—OSO₃⁻M+,

in which n stands for values from 5 to 21, preferably from 7 to 19, particularly preferably from 9 to 17, and in particular from 11 to 13, and k for values of 1, 2, 3, 4, 5, 6, 7, 8, 9, or 10, preferably for 1, 2, or 3, and in particular for 2, and M stands for a cation from the group comprising Na⁺, K⁺NH₄⁺, 1/2 Mg²⁺, 1/2 Zn²⁺, preferably for Na⁺.

The hair treatment agents can include as a further optional ingredient 0.01 to 10% by weight of at least one polymer from the group of cationic and/or amphoteric polymers.

Cationic or amphoteric polymers are understood to be polymers with a group that can be cationic “temporarily” or “permanently” in the main and/or side chain. Polymers that independent of the pH of the agent have a cationic group are designated as “permanently cationic.” These are usually polymers that include a quaternary nitrogen atom, for example, in the form of an ammonium group. Preferred cationic groups are quaternary ammonium groups. In particular, polymers in which the quaternary ammonium group is bound via a C₁-4 hydrocarbon group to a polymer main chain made up of acrylic acid, methacrylic acid, or derivatives thereof have proven to be especially suitable.

Preferred cationic polymers are selected from:
poly(methacryloyloxyethyltrimethylammonium chloride) (INCI: Polyquaternium-37), and/or
quaternized cellulose derivatives (INCI: Polyquaternium 10), and/or
cationic alkyl polyglycosides, and/or
cationized honey, and/or
cationic guar derivatives, and/or
polymeric dimethyldiallylammonium salts and copolymers thereof with esters and amides of acrylic acid and methacrylic acid, and/or
copolymers of vinylpyrrolidone with quaternized derivatives of dialkylaminoalkyl acrylate and methacrylate, and/or
vinylpyrrolidone/vinyl imidazolium methochloride copolymers, and/or
quaternized polyvinyl alcohol, and/or
Polyquaternium-2, and/or
Polyquaternium-6, and/or
Polyquaternium-7, and/or
Polyquaternium-17, and/or
Polyquaternium-18, and/or
Polyquaternium-24, and/or
Polyquaternium-27, and/or
Polyquaternium-101.

In addition to cationic polymers or instead of them, the hair treatment agents can also include amphoteric polymers. These have in addition to the cationically charged group(s) at least one negatively charged group in the molecule and are also called zwitterionic polymers.

Preferably, the polymer or polymers are used within rather narrow amount ranges. Thus, agents are preferred that, based on their weight, include 0.05 to 7.5% by weight, preferably 0.1 to 5% by weight, particularly preferably 0.2 to 3.5% by weight, and in particular 0.25 to 2.5% by weight of amphoteric polymer(s).

Regardless of whether amphoteric polymers are or are not present in the agents, further preferred agents are characterized in that, based on their weight, they include 0.05 to 7.5% by weight, preferably 0.1 to 5% by weight, particularly preferably 0.2 to 3.5% by weight, and in particular 0.25 to 2.5% by weight of cationic polymer(s).

A further group of possible ingredients are silicones. Hair treatment agents include the silicone(s) preferably in amounts of 0.1 to 10% by weight, preferably of 0.25 to 7% by weight, and in particular of 0.5 to 5% by weight, based in each case on the total agent.

Further suitable ingredients comprise nonionic polymers, fatty substances, waxes, protein hydrolysates, amino acids, vitamins, provitamins, vitamin precursors, betaines, bioquinones, purine (derivatives), taurine (derivatives), L-carnitine (salts), panthenol, pantothenic acid, 2-furanones, 2-furanone derivatives, ectoine, allantoin, plant extracts, ester oils, UV light protection filters, structuring agents, thickeners, electrolytes, pH adjusting agents, swelling agents, dyes, antidandruff agents, complexing agents, opacifiers, pearlescent agents, pigments, stabilizing agents, propellants, antioxidants, perfume oils, and/or preservatives.

It is particularly preferred that a further ingredient of the hair treatment agent is citric acid.

A further subject of the present invention is a method for reducing and/or preventing the bleeding and/or fading of artificially produced hair colors and/or for improving the color intensity and/or the color fidelity, and for improving at least one of the properties
tensile strength of keratinic fibers, in particular human hair,
stabilization of the moisture balance of keratinic fibers, in particular human hair,
combability of keratinic fibers, in particular human hair,
increase in the contact angle between water drops and the surface of keratinic fibers, in particular human hair,
reduction of the loss of elasticity of keratinic fibers, in particular human hair, in the case of damage by atmospheric effects,

in which a hair treatment agent with a pH between 3.5 and 5.5, obtainable by mixing
a) 0.00001 to 20% by weight, based on the total weight of the hair treatment agent, of at least one proteolipid of the formula (I)

R′—X—R″ (I),
where
   R′ stands for a straight-chain or branched, saturated or unsaturated hydrocarbon group having 11 to 24 carbon atoms,
   R″ denotes a protein, a peptide, or a protein hydrolysate,
   X stands for —C(O)O— or —N⁺(R^{III}₂)—R^{IV}— or —N(R^{III})R^{IV}— or —C(O)—N(R^{V})R^{VI}—,
   R^{III} denotes —(CH₂)ₓ—CH₃ with x=0-22, and
   R^{IV} denotes —CH₂—CH(OH)—CH₂— or —(CH₂)ₓ— with x=0-22;
   R^{V} and R^{VI} independently of one another stand for —H or —(CH₂)ₓ—CH₃ with x=0-22;
with the proviso that R″ stands for keratin or a keratin hydrolysate, when X stands for —C(O)O—, and
b) 0.01 to 5.0% by weight, based on the total weight of the hair treatment agent, of magnesium citrate in a water-containing carrier,
   is applied to the keratinic fibers, left there for 5 seconds to 10 minutes, and then rinsed out, or
   is applied to the keratinic fibers and remains there.

The statements made about the hair treatment agents apply mutatis mutandis to other preferred embodiments of the method.

The following examples are intended to explain the subject of the present invention but without limiting it.

### Examples

All quantitative data, unless otherwise indicated, are parts by weight of active substance. The following formulations were prepared with use of known preparation methods.

Hair rinses 1:

| | % by weight | | | |
|---|---|---|---|---|
| | 1A | 1B | 1C | 1D |
| Cetearyl alcohol | 5.0 | 5.0 | 5.0 | 5.0 |
| Quaternium-87 | 0.75 | 0.75 | 0.75 | 0.75 |
| Glycol distearate | 1.0 | 1.0 | 1.0 | 1.0 |
| Proteolipid* | 0.1 | 0.1 | 0.1 | 0.1 |
| Shea butter (INCI: | 1.5 | 1.5 | 1.5 | 1.5 |
| Butyrospermum Parkii | | | | |
| (Shea) Butter) | | | | |
| Anhydrous trimagnesium dicitrate | 1.0 | 1.5 | 1.0 | 1.0 |
| Lactic acid | 0.1 | 0.1 | 0.1 | 0.1 |
| Behenoyl PG-trimonium chloride | 1.5 | 1.5 | 1.5 | 1.5 |
| Behentrimonium chloride | 0.5 | 0.5 | 0.5 | 0.5 |
| Polyquaternium-37 | 0.4 | 0.4 | 0.4 | 0.4 |
| Amodimethicone/ | — | 0.2 | — | — |
| morpholinomethyl | | | | |
| silsesquioxane copolymer | | | | |
| Polyquaternium-101 | — | — | 0.2 | — |
| Polyquaternium 10 | — | — | — | 0.2 |
| Glycerol | 0.1 | 0.1 | 0.1 | 0.1 |
| Dimethicone | 1.0 | 0.5 | 0.5 | 1.0 |
| Water, preservative, | To 100 | To 100 | To 100 | To 100 |
| accompanying substances, and | | | | |
| optionally perfume oils | | | | |

Care spray 2:

| | % by weight |
|---|---|
| Stearamidopropyl dimethylamine | 0.3 |
| Glycerol | 2.0 |
| Polyquaternium-10 | 0.2 |
| Proteolipid* | 0.1 |
| Panthenol | 0.2 |
| Anhydrous trimagnesium dicitrate | 1.0 |
| Ethanol | 15 |
| Nicotinic acid amide | 0.1 |
| Dicaprylyl carbonate | 0.1 |
| Polyquaternium-72 | 0.2 |
| Cetrimonium chloride | 0.2 |
| Water, preservative, accompanying | To 100 |
| substances, and optionally perfume oils | |

Hair shampoos 3:

| | % by weight | | | | |
|---|---|---|---|---|---|
| | 3A | 3B | 3C | 3D | 3E |
| Sodium laureth sulfate | 9.0 | 9.0 | 9.0 | 9.0 | 9.0 |
| Ammonium lauryl sulfate | 5.0 | 5.0 | 5.0 | 5.0 | 5.0 |
| Disodium | 2.0 | 2.0 | 2.0 | 2.0 | 2.0 |
| cocoamphodiacetate | | | | | |
| Cocoamidopropyl betaine | 6.0 | 6.0 | 6.0 | 6.0 | 6.0 |
| Anhydrous | 1.5 | 1.5 | 1.5 | 1.5 | 1.5 |
| trimagnesium dicitrate | | | | | |
| Panthenol | 0.2 | 0.2 | 0.2 | 0.2 | 0.2 |
| Proteolipid* | 0.3 | 0.3 | 0.3 | 0.3 | 0.3 |
| Calcium lactate | 0.4 | 0.4 | 0.4 | 0.4 | — |
| Guar | 0.4 | 0.6 | 0.4 | 0.6 | 0.6 |
| hydroxypropyltrimonium | | | | | |
| chloride | | | | | |
| PEG-12 Dimethicone | — | 0.3 | — | — | — |
| Polyquaternium-6 | — | — | 0.5 | — | — |
| Polyquaternium-7 | — | — | — | 0.4 | — |
| Polyquaternium-67 | — | — | — | — | 0.4 |
| Sodium chloride | 1.3 | — | 1.2 | 1.3 | 1.3 |
| Water, preservative, | To 100 | To 100 | To 100 | To 100 | To 100 |
| and optionally | | | | | |
| perfume oils | | | | | |

*One of the following proteolipids A to L or a mixture of at least two of the proteolipids A to L was used as the proteolipid:

Proteolipid A (wheat protein)-O—C(O)—(CH₂)₁₀—CH₃
Proteolipid B (wheat protein)-O—C(O)—(CH₂)₁₆—CH₃
Proteolipid C (wheat protein)-N⁺(CH₃)₂—(CH₂)₁₆—CH₃
Proteolipid D H₃C—(CH₂)₁₇—N⁺(CH₃)₂—CH₂—CH(OH)—CH₂-(wheat protein)
Proteolipid E H₃C—(CH₂)₁₇—N⁺(CH₃)₂—CH₂—CH(OH)—CH₂-(wheat protein)
Proteolipid F (wheat protein)-N⁺(CH₃)₂—CH₂—CH(OH)—CH₂-cocoyl
Proteolipid G (keratin)-O—C(O)—(CH₂)₁₀—CH₃
Proteolipid H (keratin)-O—C(O)—(CH₂)₁₆—CH₃
Proteolipid I (keratin)-N⁺(CH₃)₂—(CH₂)₁₆—CH₃
Proteolipid J H₃C—(CH₂)₁₇—N⁺(CH₃)₂—CH₂—CH(OH)—CH₂-(keratin)
Proteolipid K H₃C—(CH₂)₁₇—N⁺(CH₃)₂—CH₂—CH(OH)—CH₂-(keratin)
Proteolipid L (keratin)-N⁺(CH₃)₂—CH₂—CH(OH)—CH₂-cocoyl

The pH of all conditioners, care sprays, and hair shampoos was between 4.3 and 4.5.

The agents were prepared by means of conventional methods by mixing the ingredients in a water-containing carrier and subsequent adjustment of the pH.

The following commercial products were employed:

| Commercial product | INCI | Supplier/manufacturer |
|---|---|---|
| Lanette O | Cetearyl alcohol | BASF SE |
| Varisoft W575 PG | Quaternium-87 | Evonik |
| Cutina GMS-V or Cutina | Glycol distearate | BASF SE |
| AGS | | |
| Polymer JR400 | Polyquaternium-10 | Dow |
| Dehyquart A CA | Cetrimonium chloride | BASF SE |
| Synthalen ® CR | Polyquaternium-37 | 3V Sigma |
| Quartamin BTC 131 | Behenoyl PG-trimonium chloride | Kao Chemicals |
| Genamin KDMP | Behentrimonium chloride | Clariant |
| Tego Amid S 18 | Stearamidopropyl dimethylamine | Evonik |
| Ucare ® Polymer JR 400 | Polyquaternium-10 | Dow |
| Cetiol CC | Dicaprylyl carbonate | BASF SE |
| Texapon N70 NA | Sodium laureth sulfate | BASF SE |
| Empicol AL 70 | Ammonium lauryl sulfate | Huntsman |
| Rewoteric AM 2 | Disodium cocoamphodiacetate | Evonik |
| Tego Betain F50 | Cocoamidopropyl betaine | Evonik |
| N-Hance 3196 | Guar hydroxypropyltrimonium | Ashland |
| | chloride | |
| Belsil ADM 8301 E | Amodimethicone/morpholinomethyl | Wacker |
| | silsesquioxane copolymer | |
| Deposilk Q1 | Polyquaternium-101 | Air Products |
| Xiameter OFX 0193 Fluid | PEG-12 Dimethicone | Dow |
| Merquat 100 | Polyquaternium-6 | Lubrizol |
| Merquat 550 | Polyquaternium-7 | Lubrizol |
| SoftCAT Polymer SX 400 X | Polyquaternium-67 | Dow |
| Mirustyle CP LQ | Polyquaternium-72 | Croda |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent with a pH between 3.5 and 5.5, including, in an aqueous carrier,
a) 0.00001 to 20% by weight, based on the total weight of the hair treatment agent, of at least one proteolipid of the formula (I)
R′—X—R″ (I),
wherein
R′ stands for a straight-chain or branched, saturated or unsaturated hydrocarbon group having 11 to 24 carbon atoms,
R″ denotes a protein, a peptide, or a protein hydrolysate,
X stands for —C(O)O— or —N⁺(R^{III}₂)—R^{IV}— or —N(R^{III})R^{IV}— or —C(O)—N(R^{V})R^{VI}—,
R^{III} denotes —(CH₂)ₓ—CH₃ with x=0-22,
R^{IV} denotes —CH₂—CH(OH)—CH₂— or —(CH₂)ₓ— with x=0-22, and
R^{V} and R^{VI} independently of one another stand for —H or —(CH₂)ₓ—CH₃ with x=0-22;
with the proviso that R″ stands for keratin or a keratin hydrolysate, when X stands for —C(O)O—, and
b) 0.01 to 5.0% by weight, based on the total weight of the hair treatment agent, of magnesium citrate.

2. The hair treatment agent according to claim 1, wherein the agent includes at least one proteolipid of the formula (I), in which R″ stands for keratin or a keratin hydrolysate.

3. The hair treatment agent according to claim 1, wherein the agent includes at least one proteolipid of the formula (I), in which X stands for —N⁺(CH₃)₂—CH₂—CH(OH)—CH₂— and R′ for —(CH₂)₁₁₋₂₃—CH₃.

4. The hair treatment agent according to claim 1, wherein the agent includes at least one proteolipid of the formula (I), in which X stands for —N⁺(CH₃)₂—CH₂—CH(OH)—CH₂— and R′ for —(CH₂)₁₇—CH₃.

5. The hair treatment agent according to claim 1, wherein the agent includes at least one proteolipid of the formula (I), in which X stands for —C(O)—O— and R′ for —(CH₂)₁₇—CH₃.

6. The hair treatment agent according to claim 1, wherein the magnesium citrate is selected from the group consisting of anhydrous trimagnesium dicitrate, trimagnesium dicitrate hydrate, anhydrous magnesium hydrogen citrate, magnesium hydrogen citrate hydrate, and mixtures thereof.

7. The hair treatment agent according to claim 6, wherein the magnesium citrate is anhydrous trimagnesium dicitrate.

8. The hair treatment agent according to claim 1, further including 0.05 to 7.5% by weight one or more cationic polymer(s) selected from the group consisting of
polyquaternium-37,
polyquaternium 10,
cationic alkyl polyglycosides,
cationized honey,
cationic guar derivatives,
polymeric dimethyldiallylammonium salts and copolymers thereof with esters and amides of acrylic acid and methacrylic acid,
copolymers of vinylpyrrolidone with quaternized derivatives of dialkylaminoalkyl acrylate and methacrylate,
vinylpyrrolidone/vinyl imidazolium methochloride copolymers, and/or
quaternized polyvinyl alcohol,
Polyquaternium-2,
Polyquaternium-6,
Polyquaternium-7,
Polyquaternium-17,
Polyquaternium-18,
Polyquaternium-24,
Polyquaternium-27, and
Polyquaternium-101.

9. The hair treatment agent according to claim 1, wherein the pH of the hair treatment agent is between 4.3 and 4.7.

10. A method for reducing and/or preventing the bleeding and/or fading of artificially produced hair colors and/or for improving the color intensity and/or the color fidelity, and for improving at least one of the properties
tensile strength of keratinic fibers, in particular human hair;
stabilization of the moisture balance of keratinic fibers, in particular human hair;
combability of keratinic fibers, in particular human hair;
increase in the contact angle between water drops and the surface of keratinic fibers, in particular human hair;
reduction of the loss of elasticity of keratinic fibers, in particular human hair, in the case of damage by atmospheric effects,
the method including applying to the keratinic fibers a hair treatment agent with a pH between 3.5 and 5.5 and including
a) 0.00001 to 20% by weight, based on the total weight of the hair treatment agent, of at least one proteolipid of the formula (I)
R′—X—R″ (I),
wherein
R′ stands for a straight-chain or branched, saturated or unsaturated hydrocarbon group having 11 to 24 carbon atoms,
R″ denotes a protein, a peptide, or a protein hydrolysate,
X stands for —C(O)O— or —N⁺(R^{III}₂)—R^{IV}— or —N(R^{III})R^{IV}— or —C(O)—N(R^{V})R^{VI}—,
R″ denotes —(CH₂)ₓ—CH₃ with x=0-22,
R^{IV} denotes —CH₂—CH(OH)—CH₂— or —(CH₂)ₓ— with x=0-22, and
R^{V} and R^{VI} independently of one another stand for —H or —(CH₂)ₓ—CH₃ with x=0-22,
with the proviso that R″ stands for keratin or a keratin hydrolysate, when X stands for —C(O)O—, and
b) 0.01 to 5.0% by weight, based on the total weight of the hair treatment agent, of magnesium citrate in a water-containing carrier.
